(19) United States
(12) Patent Application Publication
(10) Pub. No.: US 2018/0287046 A1
(43) Pub. Date: Oct. 4, 2018

(54) PIEZOELECTRIC DEVICE, LIQUID EJECTING HEAD, AND LIQUID EJECTING APPARATUS

(71) Applicant: SEIKO EPSON CORPORATION, Tokyo (JP)

(72) Inventors: Eiju HIRAI, Azumino (JP); Masao NAKAYAMA, Shiojiri (JP); Naoto YOKOYAMA, Matsumoto (JP); Takeshi SAITO, Matsumoto (JP); Yoshihiro HOKARI, Azumino (JP)

(21) Appl. No.: 15/923,891

(22) Filed: Mar. 16, 2018

(30) Foreign Application Priority Data
Mar. 29, 2017 (JP) 2017-064532
Dec. 25, 2017 (JP) 2017-247345

(51) Int. Cl. H01L 41/053; B41J 2/14; B41J 2/16; H01L 41/313; H01L 41/332; B06B 1/02; B06B 1/06; A61B 8/00

(52) U.S. Cl. CPC H01L 41/0536; B41J 2/14274; B41J 2/1623; B41J 2/1626; B41J 2/1612; B06B 2201/76; H01L 41/332; B06B 1/0207; B06B 1/0622; A61B 8/4483; B41J 2202/03; H01L 41/313

(57) ABSTRACT

A piezoelectric device includes a substrate in which a plurality of spaces are arranged so as to be partitioned by a plurality of walls, and a defining member defining a portion of each of the spaces in such a way as to cross between adjacent walls being among the walls and corresponding to the each of the spaces on one face of the substrate, a plurality of piezoelectric elements formed in such a way as to be each associated with a corresponding one of the spaces. a width of each of the walls denoted by a sign a, a height of each of the walls denoted by a sign b, a thickness of the defining member denoted by a sign t, and a size of each of movable regions denoted by the sign L satisfy a formula, t×L⁴/(a×b³)≤5×10⁵.

The entire disclosure of Japanese Patent Application No. 2017-064532, filed Mar. 29, 2017 and No. 2017-247345, filed Dec. 25, 2017 are expressly incorporated by reference herein.

### BACKGROUND

### 1. Technical Field

The present invention relates to an piezoelectric device, a liquid ejecting head, and a liquid ejecting apparatus that include a substrate in which a plurality of spaces are partitioned by walls, and a defining member that defines a portion of each of the spaces on one face of the substrate.

### 2. Related Art

A piezoelectric device that includes a substrate in which a plurality of spaces are formed so as to be partitioned by walls, and a defining member defining a portion of each of the spaces on one face of the substrate, and that is configured to allow portions each associated with a corresponding one of the spaces to serve as movable regions in the defining member has been applied in various apparatuses (liquid ejecting apparatuses, sensors, and the like). For example, in a liquid ejecting apparatus, a piezoelectric device configured in such a way that liquid is introduced into each of the spaces of the substrate, and a portion corresponding to the each space and included in the defining member (an elastic film) defining a portion of the each space and having flexibility is displaced by a piezoelectric element corresponding to the each space and serving as a driving element is incorporated in a liquid ejecting head (see, for example, JP-A-2017-013390). In such a liquid ejecting head, the driving of the piezoelectric element causes the portion corresponding to the each space and included in the defining member to be displaced, and this displacement of the portion of the defining member causes a pressure variation in the liquid inside the each space. Further, this pressure variation causes the liquid to be ejected through a nozzle in communication with the each space. The piezoelectric device configured in such a way as described above is sometimes employed in, for example, a sensor device and the like for detecting the pressure change, the vibration, the displacement, or the like of each of movable regions.

By the way, the above piezo electric device is constituted by a plurality of laminated materials, and thus, in the case where the individual materials have stresses (residual stresses), the stresses may cause the deformation and the distortion of the substrate during a production process. For this reason, when another member is bonded to the walls, which partition the spaces as described above, using an adhesive agent, the above-mentioned deformation and distortion of the substrate are likely to cause variations among the positions of a plurality of bonded faces of the walls and, as a result, cause a bonding failure. Further, even though, in order to reduce such a bonding failure, the thickness of the adhesive agent is further increased in view of the variation in the position of the bonded face, portions of the adhesive agent are likely to run out to the side of the above spaces from bonded regions. Further, any adhesion of the portions of the adhesive agent that have run out to the side of the spaces onto removal regions may cause the displace amounts of movable regions at the time of their displacements to be changed from those in a state before the adhesion. As a result, the displacement amounts of the movable regions are likely to vary for each of the spaces.

### SUMMARY

An advantage of some aspects of the invention is that a piezoelectric device, a liquid ejecting head, and a liquid ejecting apparatus are provided that enable the minimization of the deformation of a substrate due to a residual stress, and thereby, enable the enhancement of the reliability of the bonding of the substrate.

In a configuration of a piezoelectric device according to a first aspect of the invention, the relevant piezoelectric device includes a substrate in which a plurality of spaces are arranged in parallel so as to be partitioned by a plurality of walls, a defining member defining a portion of each of the spaces in such a way as to cross between adjacent walls being among the walls and corresponding to the each of the spaces on one face of the substrate, and a plurality of piezoelectric elements formed in such a way as to be each associated with a corresponding one of the spaces on an opposite side of the defining member from a side of the spaces, and when a width of each of the walls in a direction in which the spaces are arranged in parallel denoted by a sign a, a height of each of the walls is denoted by a sign b, the height being a size from the one face of the substrate up to another face of the substrate, the another face being on an opposite side of the substrate from the one face of the substrate, a thickness of the defining member is denoted by a sign t, and a long-length direction size of each of displaceable, movable regions in the defining member is denoted by a sign L, the width denoted by the sign a, the height denoted by the sign b, the thickness denoted by the sign t, and the size denoted by the sign L satisfy a formula, t×L⁴/(a×b³)≤5×10⁵.

According to this configuration, the strength of each of the walls in its height direction (in its vertical direction) is ensured, and thus, the deformation of the substrate is minimized. With this minimization of the deformation of the substrate, the variations among the positions of the edge faces of the walls, which are to be bonded to another material, are minimized, and thus, a bonding failure that arises when the another member is bonded to the another face of the substrate using an adhesive agent is reduced. Further, the increasing of the thickness of the adhesive agent for the purpose of the reduction of the bonding failure is made unnecessary, and thus, portions of the adhesive agent that run out (flow out) to the side of the spaces from bonded portions can be reduced. This reduction of the portions of the adhesive agent that run out to the side of the spaces minimizes the adverse influence on the displacement characteristics of the movable regions due to the phenomenon in which the portions of the adhesive agent that have flown out to the side of the spaces are adhered onto the relevant movable regions.

In the configuration of the piezoelectric device according to the first aspect of the invention, a configuration in which the width denoted by the sign a, the height denoted by the sign b, the thickness denoted by the sign t, and the size denoted by the sign L further satisfy a formula, 1.2×10⁵≤t×L⁴/(a×b³)≤1.6×10⁵ is further preferable.

According to this configuration, both of the minimization of the distortion of the substrate, and the like, and the minimization of a so-called crosstalk that causes the driving characteristics of movable regions at the time of the driving of the movable regions to be affected by each other in two adjacent spaces are achieved.

In the above configuration or in the configuration of the piezoelectric device according to the first aspect of the invention, a configuration in which the width denoted by the sign a, the height denoted by the sign b, the thickness denoted by the sign t, and the size denoted by the sign L satisfy a formula, t×b⁴/(a×L³)≤1.2×10⁻³ is further preferable.

According to this configuration, the strength of each of the walls in its space parallel-arrangement direction (in its lateral direction) is ensured, and thus, the crosstalk is minimized with certainty.

The above individual configurations are suitable configuration in a configuration in which the defining member has a residual stress.

In this configuration, the deformation of the substrate due to the residual stress of the defining member is minimized.

In the above configuration, a configuration in which a base material of the walls is silicon, and the defining material is a silicon oxide film that is formed by thermally oxidizing the base material may be employed.

According to this configuration, as the result of the binding of oxygen with the silicon, as the base material of the walls, in a process of thermal oxidization, the defining member has a compression stress (a residual stress). In this configuration, the deformation of the substrate due to the compression stress of the defining member is also minimized.

A liquid ejecting head according to a second aspect of the invention includes the piezoelectric device according to any one of the above configurations.

In the liquid ejecting head according to the second aspect of the invention, the piezoelectric device for which a bonding failure is minimized is provided, and thus, the risk of the leakage of the liquid through insufficiently bonded portions is minimized.

A liquid ejecting apparatus according to a third aspect of the invention includes the liquid ejecting head according to the second aspect of the invention.

In the liquid ejecting apparatus according to the third aspect of the invention, the liquid ejecting head for which the risk of the liquid leakage is minimized is provided, and thus, the reliability of the liquid ejecting apparatus is enhanced.

### BRIEF DESCRIPTION OF THE DRAWINGS

The invention will be described with reference to the accompanying drawings, wherein like numbers reference like elements.

FIG. 1 is a perspective view of a liquid ejecting apparatus illustrating one configuration of the liquid ejecting apparatus.

FIG. 2 is a cross-sectional view of a liquid ejecting head illustrating one configuration of the liquid ejecting head.

FIG. 3 is a cross-sectional view of a piezoelectric device in a space long-length direction illustrating one configuration of the liquid ejecting head.

FIG. 4 is a cross-sectional view of the piezoelectric device in a space parallel-arrangement direction illustrating the one configuration of the liquid ejecting head.

FIG. 5 is a plan view of a substrate illustrating one configuration of the substrate.

FIG. 6 is a schematic diagram illustrating the shape and the size of each of walls and a defining member that define a space.

FIG. 7 is a diagram illustrating a defect in a production process of the piezoelectric device.

FIG. 8 is another diagram illustrating the defect in a production process of the piezoelectric device.

FIG. 9 is a further diagram illustrating the defect in a production process of the piezoelectric device.

FIG. 10 is a graph illustrating, for each of walls corresponding to mutually different sets of dimensions, the relationship between a strength of the each wall in its vertical direction and a variation (a partition-wall height difference) in the leading edge face of the each wall.

FIG. 11 is a graph illustrating, for each of walls corresponding to mutually different sets of dimensions, the relationship between a strength of the each wall in its lateral direction and a crosstalk ratio.

FIG. 12 is a table illustrating, for each of walls having mutually different heights, a partition-wall height difference and occurrence of a crosstalk corresponding to a strength of the each wall in its vertical direction and a strength of the each wall in its lateral direction.

FIG. 13 is a plan view of a pressure-chamber forming substrate in a second embodiment of the invention.

FIG. 14 is an external view of an example of an ultrasonic diagnostic apparatus including a piezoelectric device (an ultrasonic sensor) in a third embodiment of the invention.

FIG. 15 is a plan view of the piezoelectric device in the third embodiment.

FIG. 16 is a cross-sectional view of the piezoelectric device in the third embodiment.

### DESCRIPTION OF EXEMPLARY EMBODIMENTS

Hereinafter, embodiments for practicing the invention will be described with reference to the accompanying drawings. Note that, in embodiments mentioned below, various limitations are given to configurations of the embodiments as preferred specific examples of the invention, but, in the following description, the scope of the invention is not limited to such configurations unless there is any description with particular intention of limiting the invention. Further, the following description will be made by way of exemplifying an ink jet recording head (hereinafter referred to as a recording head), as one embodiment of a liquid ejecting head including a piezoelectric device according to the invention, and exemplifying an ink jet printer (hereinafter referred to as a printer) in which the recording head is mounted, as one embodiment of a liquid ejecting apparatus in which the liquid ejecting head is mounted.

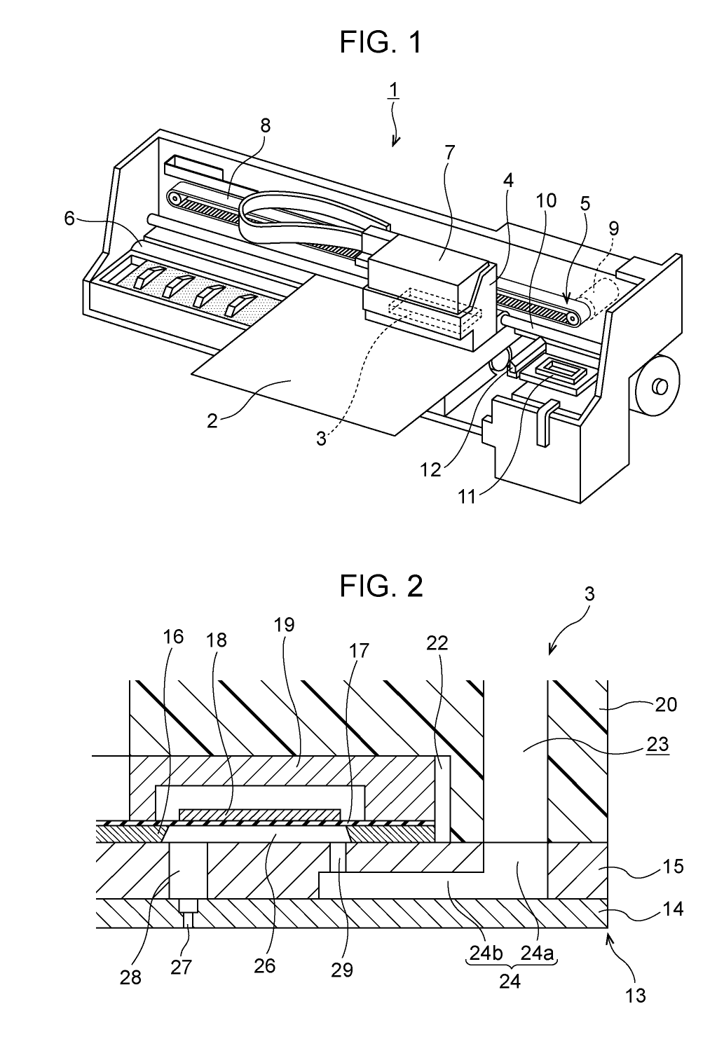

A configuration of a printer 1 will be described with reference to FIG. 1. The printer 1 is an apparatus that records an image and/or the like by ejecting ink onto the surface of a recording medium 2, such as recording paper, through nozzles 27 of a recording head 3 (see FIG. 2). The printer 1 includes the recording head 3, a carriage 4, a carriage movement mechanism 5, a platen roller 6, and the like. The recording head 3 is mounted in the carriage 4, and the carriage 4 is moved in a main-scanning direction by the carriage movement mechanism 5. The platen roller 6 feeds the recording medium 2 in a sub-scanning direction. The ink, as one kind of liquid, is stored in an ink cartridge 7, as a liquid supply source. When the ink carriage 7 is mounted into the carriage 4 in such a way as to be attachable/detachable to/from the carriage 4, the ink cartridge 7 supplies the ink stored inside itself to the recording head 3. In this case, a configuration in which the ink cartridge 7 is disposed at the body side of the printer 1 and the ink is supplied from the ink cartridge 7 to the recording head 3 through an ink supply tube can be employed.

The above carriage movement mechanism 5 includes a timing belt 8. Further, the timing belt 8 is driven by a pulse motor 9, such as a DC motor. Thus, upon actuation of the pulse motor 9, the carriage 4 reciprocates in the main-scanning direction (in a width-direction of the recording medium 2) so as to be guided by a guide rod 10. This guide rod 10 is disposed between the both side faces of the printer 1.

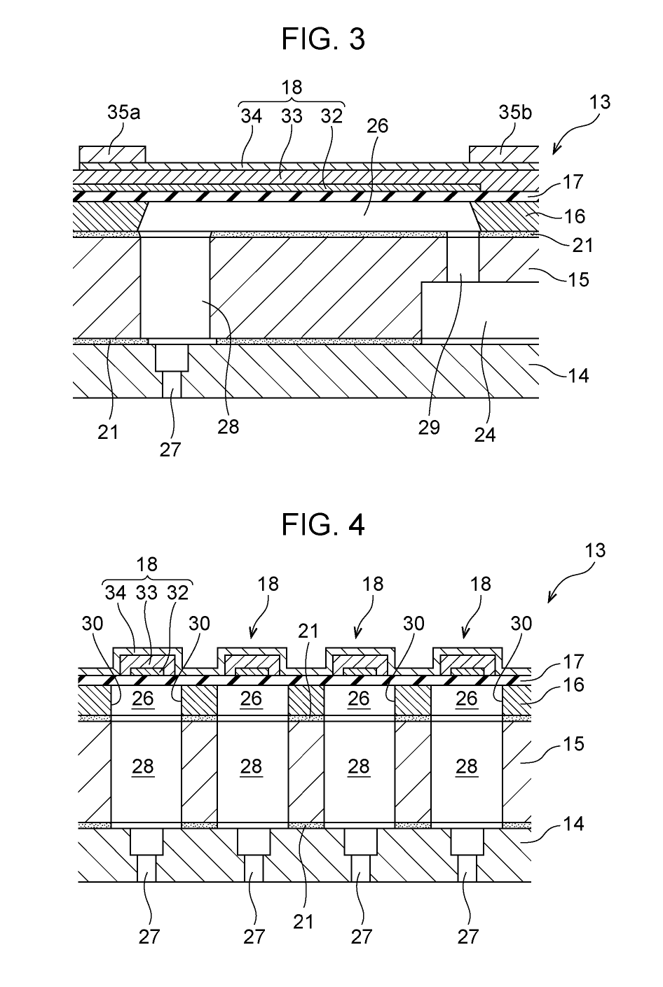

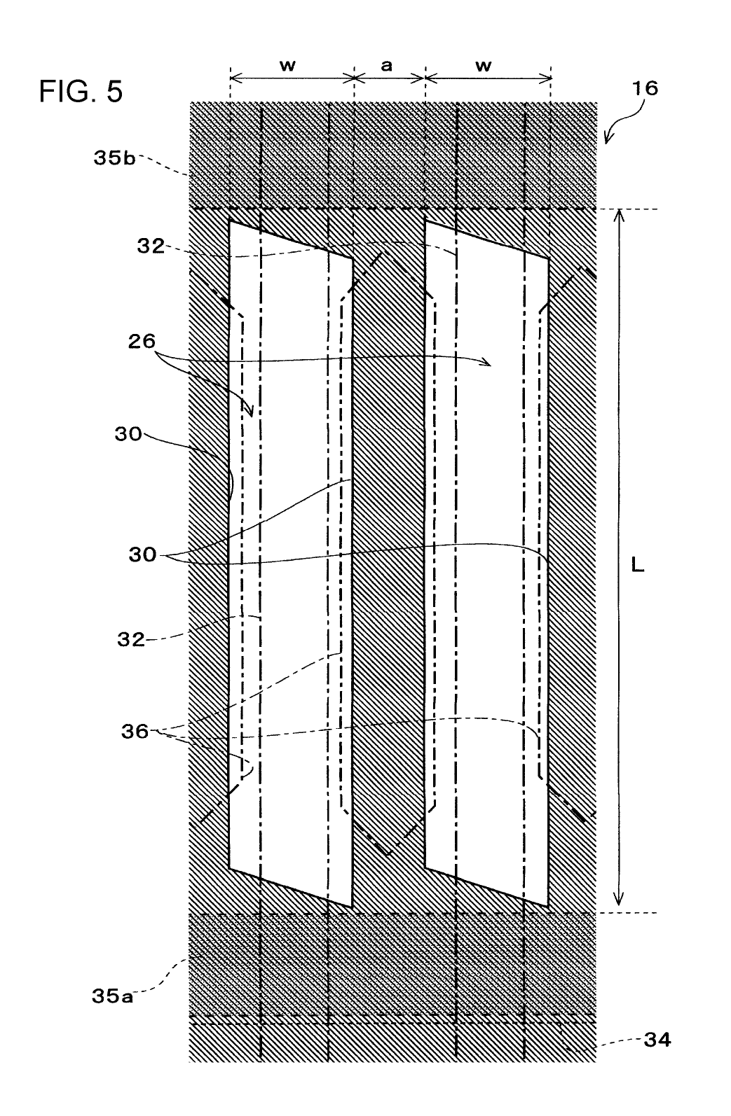

FIG. 2 is a cross-sectional view of the recording head 3 illustrating an example of the recording head 3. Further, FIG. 3 is a cross-sectional view of a piezoelectric device 13 included in the recording head 3 in a pressure-chamber long-length direction, and FIG. 4 is a cross-sectional view of the piezoelectric device 13 in a pressure-chamber parallel-arrangement direction. FIG. 5 is a plain view of the vicinity of upper openings of pressure chambers 26 in a pressure-chamber forming substrate 16. Here, for the sake of convenience, the present description is made on an assumption that a direction in which individual members are laminated corresponds to the upper-lower direction of each figure. The recording head 3 in the present embodiment includes the piezoelectric device 13, as one embodiment of a piezoelectric device according to the invention. The piezoelectric device 13 is unitized in such a way that a plurality of substrates, that is, specifically, a nozzle plate 14, a communication substrate 15, and the pressure-chamber forming substrate 16 (one kind of the substrate in the invention), are laminated in this order, and are bonded to each other using an adhesive agent 21. Further, in the piezoelectric device 13, on a face of the pressure-chamber forming substrate 16, the face being on the opposite side of the pressure-chamber forming substrate 16 from its communication substrate 15 side face, an elastic film 17 (one kind of the defining member in the invention), a piezoelectric element 18 (one kind of the driving element), and a protection element 19, as a protection element for protecting the piezoelectric element 18, are laminated. Further, the piezoelectric device 13 configured in such a way as described above is mounted into a case 20, thereby allowing the recording head 3 to be built up.

The case 20 is a synthetic-resign and box-shaped member including the piezoelectric device 13 fixed to the bottom side of the case 20. A containing hollow portion 22 is formed on the side of the lower face of the case 20, and this containing hollow portion 22 has a rectangular-solid shape that is concave from the relevant lower face up to a midway portion in a height direction of the case 20. When the piezoelectric device 13 is bonded to the lower face, the pressure-chamber forming substrate 16, and the elastic film 17, and the piezoelectric element 18, and the protection substrate 19, which are included in the piezoelectric device 13, are contained inside the containing hollow portion 22. Further, the case 20 includes an ink guide path 23 formed therein. The ink from the side of the above cartridge 7 is introduced into a common liquid chamber 24 of the piezoelectric device 13 through the ink guide path 23.

The pressure-chamber forming substrate 16 in the present embodiment is made from a silicon single-crystal substrate (corresponding to the base material in the invention, and being hereinafter referred to as just a silicon substrate). In the pressure-chamber forming substrate 16, a plurality of pressure-chamber hollow portions for defining and forming the pressure chambers 26 (one kind of the spaces in the invention) are arranged in parallel so as to be partitioned by partition walls 30 (one kind of the walls in the invention). The individual pressure-chamber hollow portions are formed by performing anisotropic etching on the pressure-chamber forming substrate 16 from the communication substrate 15 side face of the pressure-chamber forming substrate 16. This method that allows spaces that become flow paths, such as the pressure chambers, to be formed by means of the anisotropic etching on the silicon substrate enables ensuring higher accuracy in size and shape. The openings of the pressure-chamber hollow portions (hereinafter referred to as upper openings) on one face of the pressure-chamber forming substrate 16 (this face being an opposite-side face relative to its communication substrate 15 side face) are sealed by the elastic film 17. On the other hand, the communication substrate 15 (one kind of another member) is bonded to the other face of the pressure-chamber forming substrate 16, this face being on the opposite side of the pressure-chamber forming substrate 16 from the elastic film 17, and the other side openings of the pressure chamber hollow portions are sealed by the communication substrate 15. With this configuration, the pressure chambers 26 are defined and formed. That is, the elastic film 17 defines a portion of each of the pressure chambers 26 in such a way as to cross between two adjacent partition walls 30 corresponding to the each of the pressure chambers 26.

In the present embodiment, the pressure-chamber forming substrate 16 and the elastic film 17 are integrally formed. More specifically, a silicon oxide film (SiO₂) is formed by thermally oxidizing one face of the silicon substrate that is a base material of the pressure-chamber forming substrate 16. Further, the pressure-chamber hollow portions are formed by performing the anisotropic etching on the silicon substrate from the other face of the silicon substrate until the arrival at the silicon oxide film, and a remained silicon oxide film functions as the elastic film 17. Here, an unillustrated insulator film made of zirconium dioxide (ZrO₂) is laminated on the elastic film 17. Further, each of the piezoelectric elements 18 is formed at a position associated with a corresponding one of the pressure chambers 26 on the insulator film (on a face of the elastic film 17, the face being on the opposite side of the elastic film 17 from the pressure chamber 26 side). Here, a portion constituting the elastic film 17 and sealing the upper opening of each of the pressure chambers 26 functions as a portion of a movable region that is displaceable by driving of a corresponding one the piezoelectric elements 18. On the contrary, not only a region which is included in the elastic film 17 and is located outside the upper opening of each of the pressure chambers 26 and on which a weight layer 35 described later is formed, but also a region located further outside than the above region, becomes a non-movable region in which the bending deformation of the elastic film 17 is inhibited. The details of the movable region will be described later.

The pressure chamber 26 in the present embodiment is a hollow portion having a long length in a direction perpendicular to a parallel-arrangement direction of the nozzles 27. One end portion of the pressure chamber 26 in its long-length direction communicates with the nozzle 27 via a nozzle communication outlet 28 of the communication substrate 15. On the other hand, the other end portion of the pressure chamber 26 in its long-length direction communicates with a common liquid chamber 24 via a separate communication inlet 29 of the communication substrate 15. Further, a plurality of the pressure chambers 26 each associated with a corresponding one of the nozzles 27 are arranged in parallel along a nozzle-row direction so as to be partitioned by the partition walls 30.

The piezoelectric element 18 in the present embodiment is a so-called bending-mode piezoelectric element. The piezoelectric element 18 is constituted by a lower electrode layer 32 (a first electrode layer), a piezoelectric substance layer 33, as one kind of a dielectric substance, and an upper electrode layer 34 (a second electrode layer), these being laminated stepwise on the elastic film 17. In the present embodiment, the lower electrode layer 32 is independently subjected to patterning for each of the piezoelectric elements 18. As illustrated in FIG. 5, the lower electrode layer 32 has a width smaller than a width w of the pressure chamber 26 in the nozzle-row direction (in the pressure-chamber parallel-arrangement direction), and extends along the long-length direction of the pressure chamber 26. Both of the end portions of the lower electrode layer 32 in its extension direction (in its long-length direction) are provided so as to extend from the inside of the upper opening of the pressure chamber 26 up to non-movable regions outside the relevant upper opening. On the other hand, the upper electrode layer 34 is continuously formed across the individual pressure chambers 26 along the pressure-chamber parallel-arrangement direction. Further, a potion at which the upper electrode layer 34, and the piezoelectric substance layer 33, and the lower electrode layer 32 overlap with one another when seen from a lamination direction of these components is a piezoelectric-substance active portion at which an piezoelectric distortion arises in conjunction with the application of a voltage to between the both electrode layers 32 and 34. That is, the upper electrode layer 34 is used as a common electrode that is provided so as to be common to the plurality of piezoelectric elements 18; while the lower electrode layer 32 is used as a separate electrode that is separately provided for each of the piezoelectric elements 18. In addition, as each of the lower electrode layer 32 and the upper electrode layer 34, one of various kinds of metals, such as iridium (Ir), platinum (Pt), titanium (Ti), tungsten (W), nickel (Ni), palladium (Pd), and gold (Au); an alloyed metal of some of these metals; one of conductive oxidized materials, such as LaNiO₃; and the like is used.

The piezoelectric substance layer 33 in the present embodiment is formed on the elastic film 17 in a state of covering the lower electrode layer 32. As the piezoelectric substance layer 33, a material including lead (Pb), titanium (Ti), or zirconium (Zr), that is, for example, a ferroelectric piezoelectric material, such as a lead zirconate titanate (PZT) material; a material produced through the addition of metal oxide, such as niobium oxide, nickel oxide, or magnesium oxide, to the ferroelectric piezoelectric material; or the like, can be used. As illustrated in FIG. 5, an opening 36 is formed in a portion corresponding to a region between adjacent pressure chambers 26 in the piezoelectric substance layer 33. This opening 36 is constituted by a concave portion or a penetrating hole that has been formed as the result of the partial removal of the piezoelectric substance layer 33, and extends along the sides of openings (opening edges) of the pressure chambers 26. Namely, the opening 36 is a portion whose thickness is relatively smaller than that of any other portion in the piezoelectric substance layer 33, or a portion resulting from the penetration of the piezoelectric substance layer 33. Each of the both end portions of the opening 36 in the pressure-chamber long-length direction is formed in a tapered shape, that is, in a shape whose width in the pressure-chamber parallel-arrangement direction gradually narrows toward its tip portion.

Further, at a position of the upper opening of the pressure chamber 26 in a region between adjacent openings 36, a portion included in the piezoelectric substance layer 33 and having a thickness larger than that of a portion included in the piezoelectric substance layer 33 and corresponding to the opening 36 is provided in a beam shape. The beam-shaped portion of the piezoelectric substance layer 33 is provided in a portion corresponding to the piezoelectric-substance active portion. The width of the beam-shaped portion of the piezoelectric substance layer 33 in the pressure-chamber parallel-arrangement direction is slightly smaller than the width w of the pressure chamber 26 in the pressure-chamber arrangement direction. A portion included in the piezoelectric substance layer 33 and corresponding to the piezoelectric-substance active portion can be smoothly displaced by providing the above openings 36 at both pressure-chamber parallel-arrangement direction sides of the beam-shaped piezoelectric substance layer 33.

As illustrated in FIGS. 3 and 5, the weight layers 35 are provided on the upper electrode layer 34 for the piezoelectric elements 18 in the present embodiment. In the present embodiment, a weight layer 35a and a weight layer 35b are respectively formed at a position corresponding to the end portion of one side of the pressure chamber 26 and at a position corresponding to the end portion of the other side of the pressure chamber 26. In addition, as the weight layer 35, gold (Au), copper (Cu), aluminum (Al), an alloyed metal of some of these metals, or the like is used. Further, in the case where the weight layer 35 is made of gold (Au) or the like, an adhesion layer made of titanium (Ti), nickel (Ni), chromium (Cr), tungsten (W), an alloyed metal of some of these metals, or the like may be provided between the weight layer 35 and the upper electrode layer 34.

The weight layer 35a is laminated on the upper electrode layer 34 so as to be conductive with the upper electrode layer 34 in the vicinity of one side end portion of the pressure chamber 26 in the long-length direction of the pressure chamber 26, and the weight layer 35b is laminated on the upper electrode layer 34 so as to be conductive with the upper electrode layer 34 in the vicinity of the other side end portion of the pressure chamber 26 in the long-length direction of the pressure chamber 26. The weight layers 35a and 35b in the present embodiment are provided at positions slightly outside the both end portions of each of the upper openings of the pressure chambers 26 in the long-length direction of the pressure chamber 26. Providing these weight layers 35a and 35b minimizes the irregular deformations of piezoelectric elements 18 and the elastic film 17 at the time of the driving of the piezoelectric elements 18 in the vicinity of the both edges of each of the upper openings of corresponding pressure chambers 26, and thus, minimizes the damages of the piezoelectric elements 18 and the elastic film 17 in the relevant portions. In the elastic film 17 in the present embodiment, a region that falls within a region whose width is equal to the width w of the pressure chamber 26 (more strictly speaking, within a region between adjacent openings 36 in the pressure-chamber parallel-arrangement direction) and that is located between the weight layer 35a at the one side and the weight layer 35b at the other side becomes an actually displaceable movable region at the time of the driving of a corresponding piezoelectric element 18. That is, in the present embodiment, a region that is located between the weight layers 35a and 35b and that is located outside the both end portions of each of the upper openings of the pressure chambers 26 is slightly movable in conjunction with the driving of the piezoelectric element 18, and functions as part of the movable region. The both ends of these weight layers 35a and 35b in the nozzle-row direction are provided so as to, just like the upper electrode layer 34, extend up to a portion outside the region where the aggregate of pressure chambers 26 is formed. Note that each of the weight layers 35a and 35b may be provided at a position where a portion of the each of the weight layers 35a and 35b overlaps in a plan view with a corresponding end portion of the upper opening of each of the weight chambers 26 in the long-length direction of the pressure chamber 26. Further, the weight layers 35 may not be necessarily provided. In this case, an actually displaced region at the time of the driving of the piezoelectric element 18 becomes a movable region.

The communication substrate 15 is a plate material produced from the silicon substrate, just like the pressure-chamber forming substrate 16. In the communication substrate 15, a hollow portion that becomes the common liquid chamber 24 (which is also called a reservoir or a manifold) provided common to the plurality of pressure chambers 26 of the pressure-chamber forming substrate 16 is formed by means of the anisotropic etching. The common liquid chamber 24 is a hollow portion having a long size and provided along the parallel-arrangement direction of each of the pressure chambers 26. As illustrated in FIG. 2, the common liquid chamber 24 in the present embodiment is constituted by a first liquid chamber 24a and a second liquid chamber 24b. The first liquid chamber 24a is a chamber penetrating the communication substrate 15 in its plate-thickness direction. The second liquid chamber 24b is a chamber that is formed in such a way as to extend from the lower face side of the communication substrate 15 up to a midway portion in its plate-thickness direction toward the upper face side of the communication substrate 15 and that is in a state in which a thin-walled portion remains at the upper face side of the communication substrate 15. One end portion of the second liquid chamber 24b in the liquid-chamber long-length direction (this end portion being an end portion on a side farther from the nozzle 27) communicates the first liquid chamber 24a; while the other end portion of the second liquid chamber 24b in the liquid-chamber long-length direction is formed at a position overlapping with the pressure chamber 26 in a plan view. At the side of the other end portion of the second liquid chamber 24b, that is, at the side opposite the side of the first liquid chamber 24a, a plurality of the separate communication inlets 29 each penetrating the thin-walled portion and associated with a corresponding one of the pressure chambers 26 of the pressure-chamber forming substrate 16 are formed. One end of each of the separate communication inlets 29 communicates with the second liquid chamber 24b, and the other end of the each of the separate communication inlets 29 communicates with a corresponding one of the pressure chambers 26 of the pressure-chamber forming substrate 16.

The above-mentioned nozzle plate 14 is a plate material in which openings for the plurality of nozzles 27 are provided in rows. In the present embodiment, nozzle rows are configured in such a way that the plurality of nozzles 27 are provided in rows at intervals of a formation pitch corresponding to a dot formation density. The nozzle plate 14 in the present embodiment is made from the silicon substrate, and the nozzles 27 each having a cylindrical shape are formed by performing dry etching on the silicon substrate. Further, in the piezoelectric device 13 in the present embodiment, ink flow paths are each formed from the above common liquid chamber 24 up to a corresponding one of the nozzles 27 via a corresponding one of the separate communication inlets 29, a corresponding one of the pressure chambers 26, and a corresponding one of the nozzle communication outlets 28.

Further, in the recording head 3 including the piezoelectric device 13 configured in such a way as described above, in conjunction with the change of the voltage of a driving signal applied to each of the piezoelectric element 18, a piezoelectric active portion of the each piezoelectric element 18 bends and is deformed, thereby causing a corresponding one of the movable regions of the elastic film 17 to be displaced in a direction closer to a corresponding one of the nozzles 27 or in a direction farther away from the corresponding one of the nozzles 27. With this displacement, a pressure variation arises in ink inside the corresponding one of the pressure chambers 26, and through the use of this pressure variation, the ink is ejected through the corresponding one of the nozzles 27.

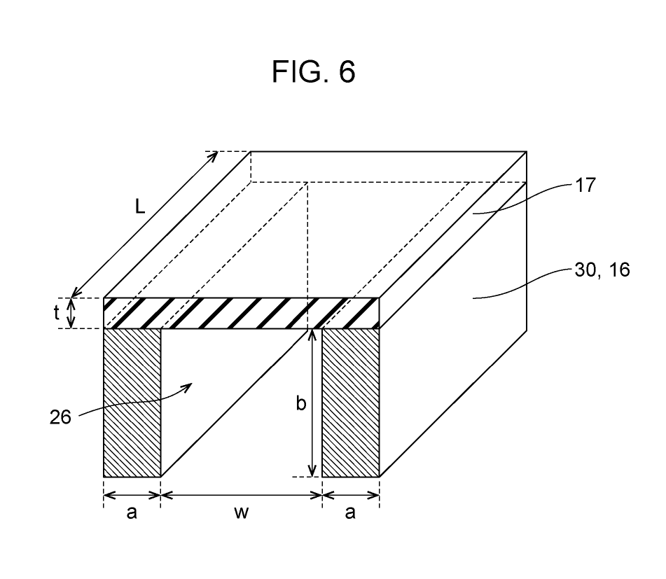

FIG. 6 is a schematic diagram illustrating the shape and the size of each of the partition walls 30 and the elastic film 17 (the movable region) that define the pressure chamber 26. Hereinafter, in the parallel-arrangement direction of the pressure chambers 26 (in the nozzle-row direction in the present embodiment), the width (thickness) of the partition wall 30 is denoted by a; the height of the partition wall 30 (the length from the base edge on the elastic film 17 side up to the leading edge on the communication substrate 15 side) is denoted by b; and the length of the movable region of the elastic film 17 in the long-length direction of the pressure chamber 26 (in the long-length direction of the movable range) is denoted by L. Recently, in response to demands for higher resolution and further miniaturization, the formation pitch for the nozzles 27 in the recording head 3 has been further narrowed, and with this narrowed formation pitch for the nozzles 27, the formation pitch for the pressure chambers 26 has been also further narrowed. Along with this trend, the width (thickness) of the partition wall 30 tends to become further thin, and the width w of the pressure chamber 26 also tends to become further narrow. When the width w of the pressure chamber 26 becomes narrow on the assumption that the size of the pressure chamber 26 in its long-length direction is constant, as a result, the capacity of the piezoelectric chamber 26 is reduced by a capacity equivalent to the narrowed portion of the width w of the pressure chamber 26. In order to maintain the weight (excluded volume) of the ink ejected through the nozzle 27 when a predetermined voltage is applied to the piezoelectric element 18, it is necessary to make the above length L of the movable region longer by making the size of the pressure chamber 26 in its long-direction direction longer. In this case, however, the strength of the partition wall 30 in its height direction is decreased by making the length L of the movable region longer. This phenomenon is likely to cause defects described below in the production process of the recording head 3 (the piezoelectric device 13).

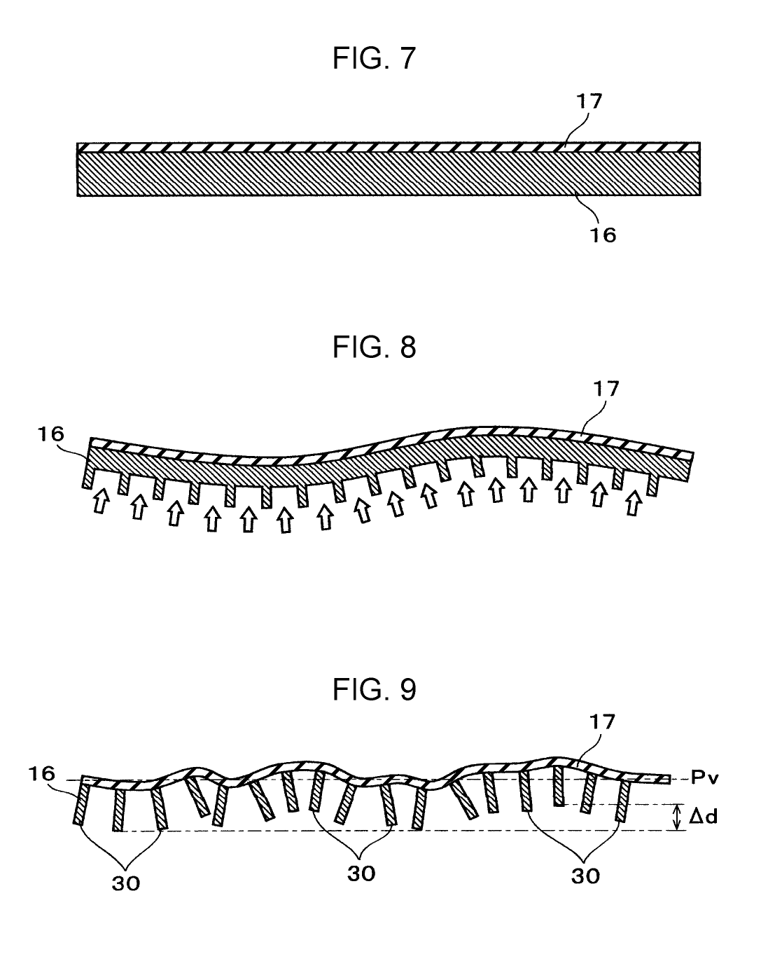

FIGS. 7 to 9 are schematic diagrams illustrating a defect in a production process of the recording head 3 (the piezoelectric device 13). Here, in each of FIGS. 7 to 8, the illustration of other constituent members, such as the piezoelectric element 18, formed on the elastic film 17 is omitted. Further, in FIGS. 8 and 9, the distortion of the pressure-chamber forming substrate 16 is more exaggeratingly illustrated than an actual one. For example, in a process of producing the piezoelectric device 13 in the present embodiment, as illustrated in FIG. 7, one face of the pressure-chamber forming substrate 16, made from the silicon substrate, is thermally oxidized, and thereby, the elastic film 17, made of the silicon oxide film, is formed. In this process, as the result of the binding of the silicon with oxygen, the silicon being a base material, the silicon attempts to expand, and as a result, the elastic film 17 has a compression stress (one kind of residual stresses). In this state, as denoted by arrows in FIG. 8, along with proceeding of a process in which hollow portions that become the pressure chambers 26 are gradually formed from the other face of the pressure-chamber forming substrate 16 by means of anisotropic etching using etching liquid containing potassium hydroxide (KOH) or the like, the strength of the pressure-chamber forming substrate 16 supporting the elastic film 17 is decreased, thereby causing the compression stress of the elastic film 17 to be gradually released, and as a result, causing a deformation (a distortion) to arise in the pressure-chamber forming substrate 16. Further, as illustrated in FIG. 9, when the anisotropic etching is performed until the arrival at the elastic film 17 serving as an etching stop, portions supporting the elastic film 17 do not exist anymore in portions corresponding to the pressure chambers 26 in the pressure-chamber forming substrate 16, and thus, the distortion that arises in the pressure-chamber forming substrate 16 and that depends on the strength of the partition wall 30 in its height direction (in the vertical direction) becomes larger. As a result, the positions of the leading edge faces of the partition walls 30, that is, the positions of faces to be bonded to the communication substrate 15, as another member, in a subsequent process, vary to a great degree. In such a state, when the bonding to the communication substrate 15 is performed, it is difficult to evenly apply the adhesive agent 21 to the leading edge faces of the partition walls 30 by means of transcription or the like, and this difficulty is likely to cause a bonding failure.

Further, the variations among the positions of the bonded faces not only cause the above-mentioned difficulty that leads to a situation where the transcription itself becomes unavailable (namely, a situation where portions incapable of being bonded occurs), the occurrence of this situation being dependent on a kind of used adhesive agent, but also, even though the transcription is available, requires the increasing of the thickness of the used adhesive agent for the purpose of the reduction of the leakage of the ink through bonded portions. Further, when the bonding is performed in such a case where the thickness of the used adhesive agent is increased, as a result, the amounts of portions of the adhesive agent that run out to the sides of the flow paths (the sides of the pressure chambers 26) vary. For example, when such portions of the adhesive agent that have run out to the sides of the flow paths travel on the partition walls 30 of the pressure chambers 26 by capillary action; reach the elastic film 17; and become hardened, the hardened portions of adhesive agent are likely to change the displacement amounts of the movable regions at the time of the driving. Further, along with the variations among the amounts of the portions of the adhesive agent that run out, the amounts of portions of the adhesive agent that creep up to the elastic film 17 also vary, and thus, as the result of the variations among the amounts of the creeped-up portions of the adhesive agent, a problem in that the displacement amounts of the movable regions vary among the individual pressure chambers 26 occurs.

In view of the above problem, in the printer 1 according the embodiment of the invention, the occurrence of the deformation of the pressure-chamber forming substrate 16 is minimized by appropriately setting the above individual dimensions. More specifically, with respect to the width a of the partition wall 30, the height b of the partition wall 30, the length L of the movable region of the elastic film 17, and the film thickness t of the elastic film 17, the individual dimensions are set so as to satisfy the following condition (1).

t×L⁴/(a×b³)≤5×10⁵ (1)

In the above condition (1), “t×L⁴/(a×b³)” is an expression representing the height-direction (vertical-direction) strength of the partition wall 30 serving as a beam whose both ends are fixed in the pressure-chamber forming substrate 16 (the expression being hereinafter referred to as a vertical strength calculating expression when needed). In the vertical strength calculating expression, the height b of the partition wall 30 is expressed by the third power of b, and the length L of the movable region is expressed by the fourth power of L, and thus, in order to ensure the vertical-direction strength of the partition wall 30, it is effective to particularly make the length L of the movable region shorter, and make the height b of the partition wall 30 lower.

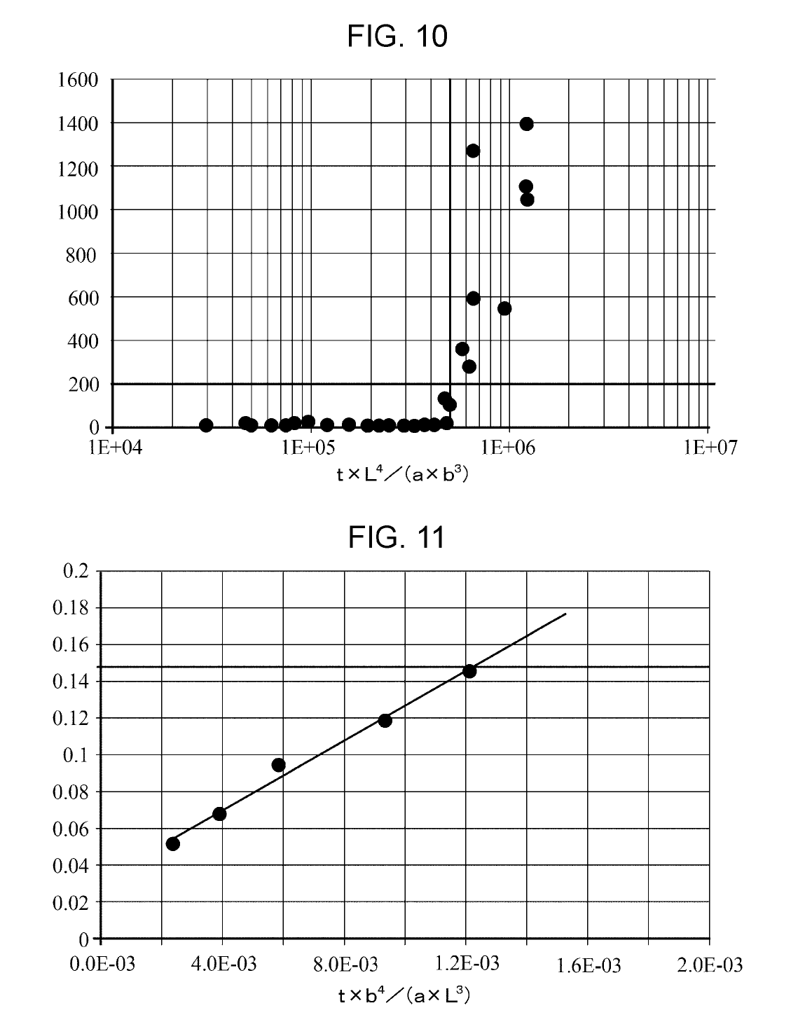

FIG. 10 is a graph illustrating, for each of the partition walls 30 included in a plurality of recording heads (piezoelectric devices) having mutually different sets of the above dimensions and mutually different shapes, the relationship between a strength of the each partition wall 30 in its vertical direction and a variation (hereinafter referred to as a partition-wall height difference) in the leading edge face of the each partition wall 30. In FIG. 10, the horizontal axis represents a calculated value of the above vertical strength calculating expression representing the vertical-direction strength of the partition wall 30, and the vertical axis represents the partition wall height difference [nm]. Here, as illustrated in FIG. 9, the partition-wall height difference is represented by a difference Δd that is the difference between a maximum value and a minimum value among the values of heights from an assumed face passing through the both edges of the pressure-chamber forming substrate 16 in the pressure-chamber parallel-arrangement direction up to the leading edge faces of the individual partition walls 30 (the assumed face being a height-direction face denoted by Pv in FIG. 9, the heights being normal-direction distances relative to the assumed face Pv).

As illustrated in FIG. 10, when the value of the vertical strength calculating expression exceeds a certain constant value, the vertical-direction strength of the partition wall 30 decreases, and the partition-wall height difference significantly increases. Here, in the case where the leading edge faces of the partition walls 30 are bonded to the communication substrate 15 by applying the adhesive agent 21 onto the leading edge faces of the partition walls 30 by means of the transcription, in order to minimize the occurrence of the bonding failure in a way that does not make the thickness of the adhesive agent 21 extremely large, that is, in a way that reduces the portions of the adhesive agent 21 that run out to the side of the flow paths, such as the pressure chambers 26, the partition-wall height difference is preferable to be smaller than or equal to 200 [nm]. Further, in order to make the partition-wall height difference smaller than or equal to 200 [nm], the value of the vertical strength is necessary to be smaller than or equal to 5×10⁵. As illustrated in FIG. 10, when the value of the vertical strength exceeds 5×10⁵, the partition-wall height difference becomes larger than 200 [nm], and this situation increases the possibility that the bonding failure occurs.

The vertical-direction strength of the partition wall 30 is ensured by setting the individual dimensions in such a way that the individual dimensions satisfy the above condition (1), and thus, the deformation of the pressure-chamber forming substrate 16 is minimized. With this minimization of the deformation of the pressure-chamber forming substrate 16, the variations among the positions of the leading edge faces of the partition walls 30 are minimized, and thus, the bonding failure that occurs when the communication substrate 15 is bonded to the other face of the pressure-chamber forming substrate 16 using the adhesive agent 21 is reduced. As a result, the yield ratio of the piezoelectric device 13 is enhanced, and its reliability is also enhanced. Particularly, in the present embodiment, the elastic film 17 has a compression stress (a residual stress) because the elastic film 17 is formed by the thermal oxidization of silicon, and thus, even when the pressure chambers 26 are formed by means of the anisotropic etching in such a state, the above-described configuration is suitable because the above-described configuration enables the minimization of the deformation of the pressure-chamber forming substrate 16, which is caused by the release of the compression stress of the elastic film 17. Further, the above-described configuration makes the increasing of the thickness of the adhesive agent 21 for the purpose of the reduction of the bonding failure unnecessary, and thus, the above-described configuration enables the reduction of the portions of the adhesive agent 21 that run out (flow out) into the flow paths, such as the pressure chambers 26. This reduction of the portions of the adhesive agent 21 that run out into the flow paths minimizes the adverse influence on the displacement characteristics of the movable regions of the elastic film 17 due to the phenomenon in which the portions of the adhesive agent 21 that have flown out to the flow path sides are adhered onto the relevant movable regions.

For the recording head 3 in which such the piezoelectric device 13 is employed, the risk of the leakage of the ink through portions where the bonding is insufficient is reduced. Further, the variations among the displacement characteristics of the movable regions due to the portions of the adhesive agent 21 that have flown out are minimized, and thus, the minimization of the variations among the ejection characteristics of the individual nozzles 27 is achieved. Moreover, the reliability of the printer 1 including such the recording head 3 is enhanced.

By the way, even when the condition (1) is satisfied, in the case where the strength of the partition wall 30 in the pressure-chamber parallel-arrangement direction (namely, the lateral-direction strength of the partition wall 30) is insufficient, the deformation of the partition wall 30 by a pressure variation that, because of the insufficient lateral-direction strength of the partition wall 30, arises inside the pressure chamber 26 at the time of the ejection of the ink is likely to cause the variations among the ejection characteristics, such as the amounts and the flying speeds of inks ejected through the respective nozzles 27, that is, a so-called crosstalk that, in adjacent spaces, causes driving characteristics of the movable regions at the time of the driving of the movable regions to be affected by each other.

Considering from this viewpoint, in the printer 1 according to the embodiment of the invention, the individual dimensions are set so as to further satisfy the following condition (2).

t×b⁴/(a×L³)≤1.2×10⁻³ (2)

In the above condition (2) , “t×b⁴/(a×L³)” is an expression representing the lateral-direction (pressure-chamber parallel-arrangement direction) strength of the partition wall 30 serving as a beam whose both ends are fixed in the pressure-chamber forming substrate 16 (the expression being hereinafter referred to as a lateral-strength calculating expression as needed).

FIG. 11 is a graph illustrating, for each of the partition walls 30 corresponding to mutually different sets of dimensions, the relationship between a strength of the each wall in its lateral direction and a crosstalk ratio. Here, the crosstalk ratio is the degree of the change of the ejection characteristic, and is represented by the ratio of an ink flying speed Vms at the time when ink is singularly ejected through one nozzle 27 (at the time of one ON) relative to an ink flying speed Vma at the time when inks are simultaneously ejected through a plurality of the nozzles 27 that are arranged adjacent to each other and belong to the same nozzle row (at the time of all ON). The crosstalk ratio is represented by the following formula (CT).

CROSSTALK RATIO=1−Vms/Vma (CT)

For example, when Vma=10 [m/s], and Vms=8 [ms], a resulting crosstalk ratio is 0.2. In the case where, in the printer 1, images and the like are recorded on the recording medium 2, and particularly, the images are required to be recorded with higher resolution, a crosstalk ratio in this case is required to be smaller than 0.15. This is because, in the case where the crosstalk ratio exceeds 0.15, on the recording medium 2, deviations from target positions with respect to the landing positions of the inks having been ejected through the nozzles 27 become significant, and as a result, for recorded images and the like, visual roughness, such as a so-called granular feeling, stands out.

Accordingly, since the lateral-direction (pressure-chamber parallel-arrangement direction) strength of the partition wall 30 is ensured by setting the individual dimensions in such a way that the individual dimensions to satisfy the above condition (2), the displacement of the partition wall 30, which is caused by the pressure variation that arises inside the pressure chamber 26 when the piezoelectric element 18 is driven to cause the ink to be ejected through the nozzle 27, is minimized. As a result, the above crosstalk is minimized. That is, the variations among the ejection characteristics (the amounts and the flying speeds of the inks ejected through the nozzles 27) are minimized.

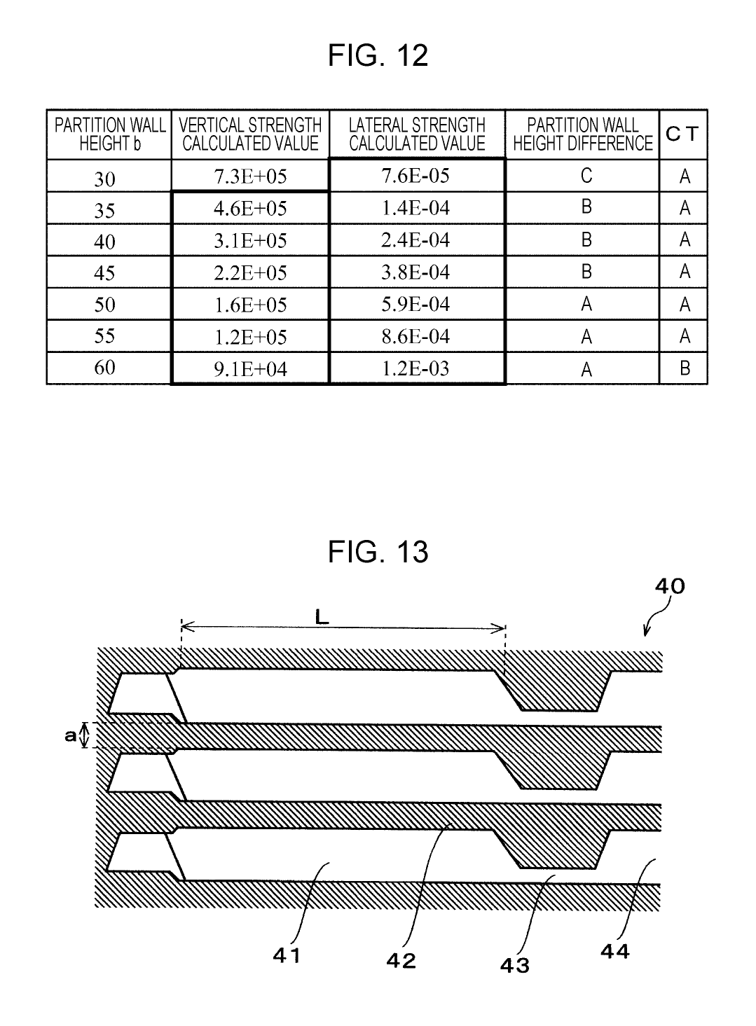

FIG. 12 is a table illustrating, for each of partition walls 30 having mutually different heights, the partition-wall height difference and the occurrence of a crosstalk (CT) corresponding to the vertical strength of the each partition wall 30 and the lateral strength of the each partition wall 30. In FIG. 12, a left-side portion enclosed by a thick-bordered box and a right-side portion enclosed by a thick-bordered box respectively indicate a range including vertical strengths that satisfy the above condition (1) and a range including lateral strengths that satisfy the above condition (2). Further, for the partition-wall height difference, “A” means that almost no partition-wall height difference occurs; “B” means that partition-wall height differences smaller than or equal to 200 [nm] occur; and “C” means that one or more partition-wall height differences larger than 200 [nm] occur. Moreover, for the crosstalk, “A” means that almost no crosstalk occurs (for example, a crosstalk ratio is smaller than 0.1), and “B” means that one or more crosstalks occur, but these crosstalks fall within an allowable range (for example, one or more crosstalk ratios fall within a range larger than or equal to 0.1 and smaller than or equal to 0.15). Further, the width a of the partition wall 30, the length L of the movable region, and the film thickness t of the elastic film 17 are fixed to values that allow the above excluded volume to be equal to a predetermined value, and only the height b of the partition wall 30 is changed to change the values of the vertical strength and the lateral strength.

As illustrated in FIG. 12, for any vertical strength that satisfies the condition (1), each of a corresponding partition-wall height difference and a corresponding crosstalk becomes “A” or “B”, and both of the minimization of the partition-wall height difference and the minimization of the crosstalk can be almost achieved. Further, for any vertical strength that satisfies the condition (1), in order to achieve a situation in which both of a corresponding partition-wall height difference and a corresponding crosstalk become “A”, this situation can be achieved merely by allowing any one of the following conditions (3) and (4) to be further satisfied.

1.2×10⁵≤t×L⁴/(a×b³)≤1.6×10⁵ (3)

5.9×10⁻⁴≤t×b⁴/(a×L³)≤8.6×10⁻³ (4)

With this configuration, both of the minimization of the partition-wall height difference and the minimization of the crosstalk can be effectively achieved.

By the way, the invention is not limited to the aforementioned embodiment, and various modifications can be made on the basis of appended claims.

FIG. 13 is a plan view of a pressure-chamber forming substrate 40 in a second embodiment of the invention. In the above first embodiment, the configuration in which the circumference of the upper opening of the pressure chamber 26 is seamlessly surrounded by the structured wall including the partition walls 30 of the pressure-chamber forming substrate 16 is exemplified, but the configuration of a pressure-chamber forming substrate is not limited to such a configuration. In the pressure-chamber forming substrate 40 in the present embodiment, not only a plurality of pressure chambers 41 are arranged in such a way as to be partitioned by partition walls 42, but also a common liquid chamber 44 and separate communication inlets 43 are formed. The pressure chambers 41 correspond to the spaces in the invention, and the partition walls 42 correspond to the walls in the invention. The common liquid chamber 44 is provided common to the pressure chambers 41, and each of the separate communication inlets 43 allows the common liquid chamber 44 to communicate with a corresponding one of the pressure chambers 41. With this configuration, the entire circumference of the upper opening of the pressure chamber 41 is not closed by a structured wall of the pressure-chamber forming substrate 40 including the partition walls 42, but opens at a portion corresponding to the separate communication inlet 43. The invention can be also applied to this configuration, as well as to the above configuration of the first embodiment. That is, when the width of the partition wall 42 in a parallel-arrangement direction of the pressure chambers 41 is denoted by a; the height of the partition wall 42 is denoted by b; the length of each of movable regions of an elastic film (a defining member) provided on one face of the pressure-chamber forming substrate 40 is denoted by L; and the thickness of the elastic film is denoted by t, in the case where the above individual dimensions are set so as to satisfy the above conditions (1) to (4), the same advantageous effects as those of the above first embodiment are brought about. In addition, the other configurations are the same as those of the first embodiment.

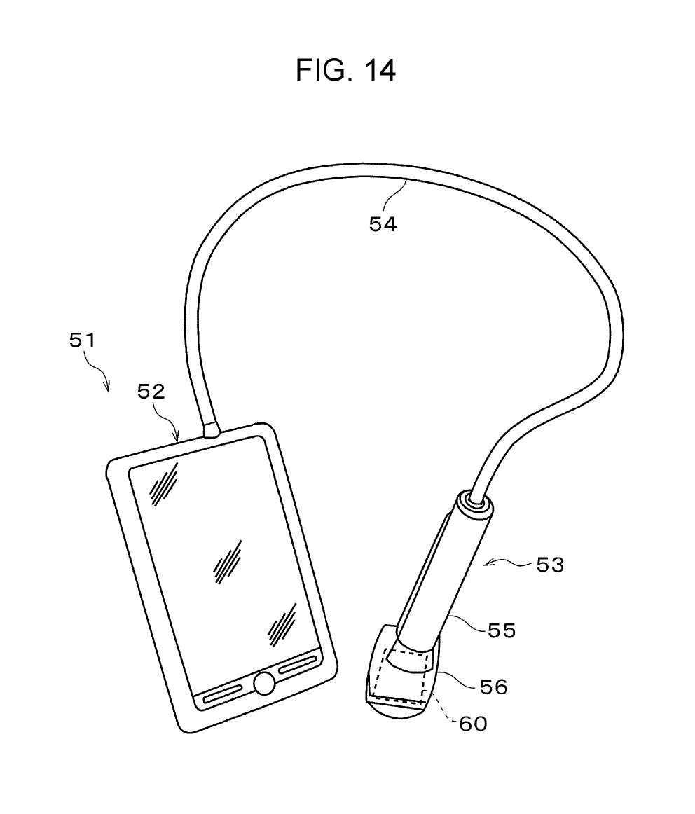

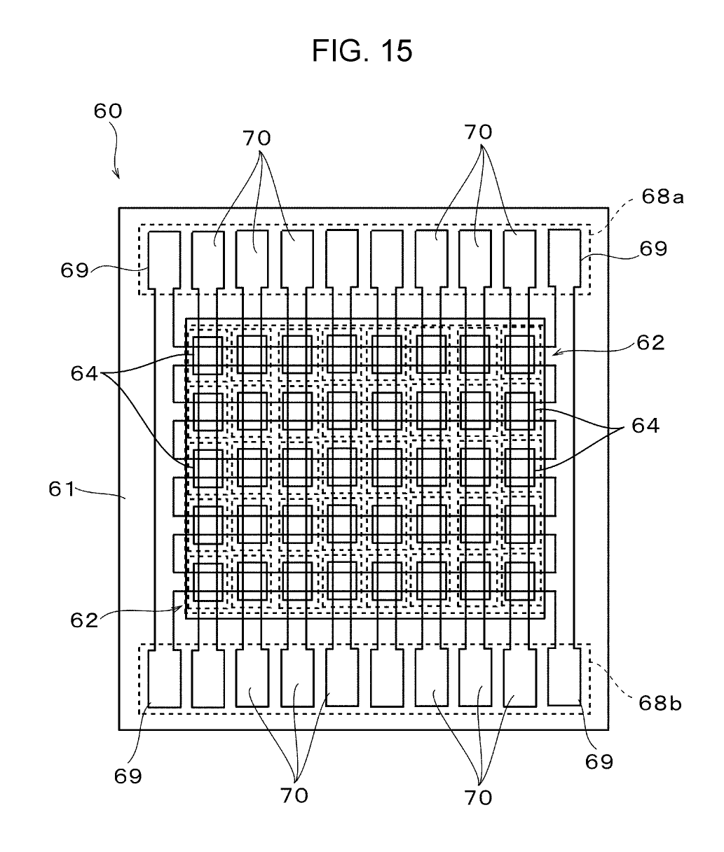

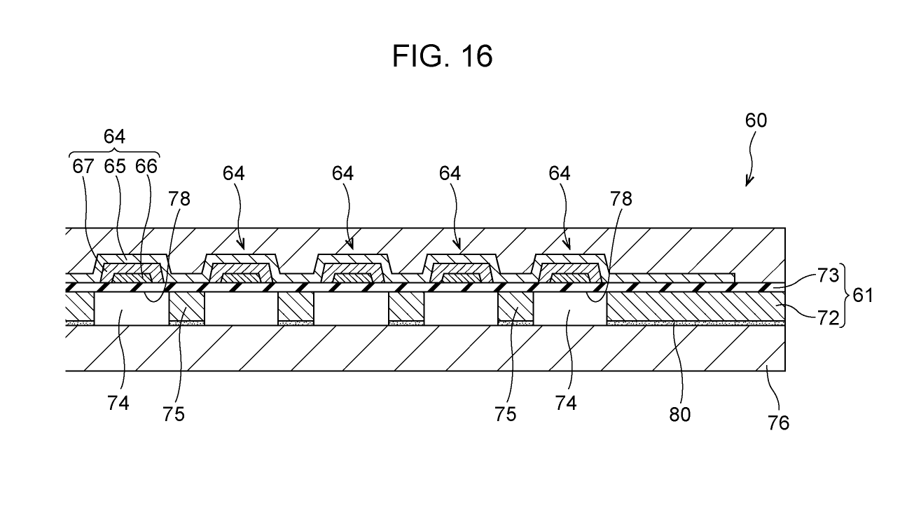

FIG. 14 is a diagram illustrating a configuration of an ultrasonic diagnostic device 51 including an ultrasonic sensor 60, as one kind of electronic device in a third embodiment. Further, FIG. 15 is a plan view of an example of the ultrasonic sensor 60 according to the present embodiment, and FIG. 16 is a fragmentary cross-sectional view of the ultrasonic sensor 60 in a row direction of the ultrasonic sensor 60 (namely, in the horizontal direction in FIG. 15). In the above individual embodiments, the configuration in which ink, as one kind of liquid, is ejected through a nozzle upon displacement of a corresponding movable region has been exemplified, but without being limited to this configuration, the invention can be also applied to a sensor or the like, such as the ultrasonic sensor 60 in the present embodiment, for detecting a vibration (a displacement) of a movable region. Thus, the spaces in the invention are not limited to objects through which the liquid is flown.

The ultrasonic diagnostic device 51 illustrated in FIG. 14 includes a device terminal 52 and an ultrasonic probe 53. The device terminal 52 and the ultrasonic probe 53 are interconnected by a cable 54. The device terminal 52 and the ultrasonic probe 53 transmit/receive electric signals through the cable 54. The ultrasonic probe 53 includes a body portion 55 and a probe head 56. This probe head 56 is attachably/detachably mounted into the body portion 55. Further, the ultrasonic sensor 60 is mounted in the probe head 56. The ultrasonic sensor 60 detects the distance up to a measurement object, the shape of the measurement object, and the like by transmitting a sonic wave (an ultrasonic wave) toward the measurement object from the surface of the ultrasonic sensor 60 (from the face illustrated in FIG. 15) and receiving reflected waves from the measurement object. The ultrasonic sensor 60 in the present embodiment is configured such that an element array 62 is formed on a base body 61. The element array 62 is constituted by an array of piezoelectric elements 64. The array is formed in a matrix of a plurality of rows and a plurality of columns. As illustrated in FIG. 16, each of the piezoelectric elements 64 is constituted by an upper electrode 65, a lower electrode 66, and a piezoelectric substance film 67, and the piezoelectric substance film 67 is interposed between the upper electrode 65 and the lower electrode 66. In the present embodiment, the upper electrode 65 functions as a common electrode common to the individual piezoelectric elements 64, and the lower electrode 66 functions as a separate electrode corresponding to each of the piezoelectric elements 64. Here, the function of the upper electrode 65 and the function of the lower electrode 66 may be interchanged. That is, a lower electrode may be provided common to the piezoelectric elements 64 of the entire matrix; while an upper electrode may be separately provided for each of the piezoelectric elements 64. Further, for the array of the element array 62, for example, a configuration in which, for any pair of adjacent columns, the column-direction positions of piezoelectric elements 64 belonging to one column of the pair of columns and the column-direction positions of piezoelectric elements 64 belonging to the other column of the pair of columns are disposed so as to alternately appear along with movement of an observation position in the column direction may be employed. In this case, a group of piezoelectric elements 64 belonging to any one of even-number columns can be configured to be arranged so as to be shifted by half of a row pitch in the column direction, relative to a group of piezoelectric elements 64 belonging to any one of odd-number columns.

On the base body 61, a first terminal array 68a and a second terminal array 68b are each formed at a position located outside the element array 62 and associated with a corresponding one of one end side and the other side in the column direction of the piezoelectric elements 64. Each of the terminal arrays 68a and 68b is constituted by a pair of common electrode terminals 69 and a plurality of separate electrode terminals 70. The common electrode terminals 69 are arranged at both sides in the row direction, and the plurality of separate electrode terminals 70 are arranged between the common electrode terminals 69. The common electrode terminals 69 are conductive with the upper electrodes 65 of the piezoelectric electrodes 64, and the separate electrode terminals 70 are conductive with the lower electrodes 66 of the piezoelectric electrodes 64. Each of the terminal arrays 68a and 68b is electrically connected to an unillustrated flexible wiring substrate whose one end is connected to an unillustrated control circuit of the ultrasonic diagnostic device 51. Through the flexible wiring substrate, as described later, a driving signal VDR and received signals VR are transmitted/received between the control circuit and the ultrasonic sensor 60.

As illustrated in FIG. 16, the base body 61 includes a substrate 72 and a flexible film 73 (one kind of the defining member in the invention) in a state in which these substrate 72 and flexible film 73 are laminated each other. More specifically, the flexible film 73 is formed all over one face of the substrate 72. On the substrate 72, a plurality of hollow portions 74 (one kind of the spaces in the invention) each associated with a corresponding one of the piezoelectric elements 64 are defined and formed by partition walls 75 (one kind of the walls in the invention). That is, the hollow portions 74 are disposed in the form of an array relative to the substrate 72, and one of the partition walls 75 is disposed between every two adjacent ones of the hollow portions 74. Further, a portion included in the flexible film 73 and corresponding to an upper opening of each of the hollow portions 74 functions as a corresponding one of movable regions 78. Each of the movable regions 78 is a portion that is included in the flexible film 73 and defines one portion (a ceiling face) of a corresponding one of the hollow portions 74 and that is vibratable (displaceable) in a thickness direction of the substrate 72. In the present embodiment, the substrate 72 and the flexible film 73 are integrally formed. More specifically, a silicon oxide film (SiO₂) is formed by thermally oxidizing one face of a silicon substrate, as a base material of the substrate 72. Further, the hollow portions 74 are formed by performing anisotropic etching on the silicon substrate from the other face of the silicon substrate until the arrival at the silicon oxide film, and a remained silicon oxide film functions as the flexible film 73. Here, an unillustrated insulator film is laminated on the flexible film 73. Further, each of the piezoelectric elements 64 is disposed at a predetermined position by stepwise laminating the lower electrode 66, the piezoelectric substance film 67, and the upper electrode 65 on a surface of a corresponding one of the movable regions (the surface being its face on the opposite side relative to its face on the hollow portion 74 side).

A reinforcing plate 76 is bonded to a reverse face of the base body 61 by an adhesive agent 80 (the reverse face being its face on the opposite side relative to its face on the flexible film 73 side). The reinforcing plate 76 closes the hollow portions 74 on the reverse side of the ultrasonic sensor 60. As the reinforcing plate 76, for example, a silicon substrate can be used.

In the above ultrasonic sensor 60, during a transmitting period (a vibration period) when an ultrasonic wave is transmitted, the driving signal VDR output by the control circuit is supplied (applied) to the lower electrodes 66 of the piezoelectric elements 64 via the separate electrode terminals 70. During a receiving period (the vibration period) when reflected waves (echoes), as ultrasonic waves, are received, the received signals VR from the piezoelectric elements 64 are output via the lower electrodes 66 and the separate electrode terminals 70. Further, a common voltage VCOM is supplied to the upper electrodes 65 of the piezoelectric elements 64 via the common electrode terminals 69. This common voltage VCOM is a constant direct-current voltage. When a difference voltage between the driving signal VDR and the common voltage VCOM is applied to each of the piezoelectric elements 64, an ultrasonic wave having a predetermined frequency is transmitted from the each of the piezoelectric elements 64. Further, the ultrasonic wave radiated from each of all the piezoelectric elements 64 is synthesized, and thereby, an ultrasonic wave radiated from the element-array face of the ultrasonic sensor 60 is formed. This ultrasonic wave is transmitted toward a measurement object (for example, the inside of a human body). Further, after the transmission of the ultrasonic wave, upon input of a reflected wave having been reflected from the measurement object into one of the piezoelectric elements 64, in response to the input, the relevant piezoelectric element 64 vibrates as a detecting vibration portion, thereby causing an electromotive force to be generated. This electromotive force is output to the control circuit as one of the received signals VR. In the present embodiment, the group of piezoelectric elements serving as detecting vibration portions alternately performs the transmission of the ultrasonic wave and the reception of the reflected waves.

Just like the above first embodiment, in the case where the deformation and the distortion of the substrate 72 arise in a production process, the positions of bonded faces (faces bonded to the reinforcing plate 76 of the substrate 72) vary, and this variation in the position of the bonded face is likely to cause a bonding failure and the variation in the displacement amount of the movable region 78. Thus, in the present embodiment, when the width of the partition wall 75 is denoted by a; the height of the partition wall 75 is denoted by b; the long-length direction length of a movable region included in the removable region 78 is denoted by L, and the film thickness of the movable region 78 is denoted by t, in the case where the above individual dimensions are set in such a way that the individual dimensions satisfy the above conditions (1) to (4), the same advantageous effects as those of the above first embodiment are brought about. That is, the deformation of the substrate 72 is minimized, and thus, a bonding failure that arises when the reinforcing plate 76 is bonded to the other face of the substrate 72 using the adhesive agent 80 is reduced. As a result, the yield ratio of the ultrasonic sensor 60, as the piezoelectric device, is enhanced, and its reliability is also enhanced. Further, the increasing of the thickness of the adhesive agent 80 for the purpose of the reduction of the bonding failure is made unnecessary, and thus, portions of the adhesive agent 80 that run out (flow out) to the side of the hollow portions 74 can be reduced. This reduction of the portions of the adhesive agent 80 that run out to the side of the hollow portions 74 minimizes the adverse influence on the displacement characteristics (vibration characteristics) of the movable regions 78 due to the phenomenon in which the portions of the adhesive agent 80 that have flown out to the side of the hollow portions 74 are adhered onto the relevant movable regions 78. Moreover, the strength of each of the partition walls 75 is ensured, and thus, when a corresponding piezoelectric element 64 and a corresponding movable region 78 vibrate, the deformation of the each of the partition walls 75 is minimized. As a result, the variations among the vibration characteristics (the transmission characteristics and the reception characteristics) of the movable regions 78 due to so-called adjacent crosstalks are minimized.

Note that, with respect to the residual stress included in the defining member, in the case where a different film forming method is employed, not only the compression stress but also a tensile stress may be included in the relevant defining member, and in such a configuration, the invention can be also applied. This application of the invention to such a configuration enables the minimization of the deformation of the substrate due to the tensile stress.

Moreover, the invention can be also applied to a configuration in which each of a substrate and another member (another base material) bonded to the substrate has a residual stress (regardless of whichever this residual stress is the compression stress or the tensile stress). This application of the invention to such a configuration enables the minimization of the deformation of the substrate due to the difference between these kinds of residual stresses.

Further, although, in the above first embodiment, the description has been made by exemplifying the ink jet recording head 3 as the liquid ejecting head, the invention can be applied to any other liquid ejecting head for which a configuration in which a plurality of spaces partitioned by walls are formed as the result of bonding of a plurality of substrates using an adhesive agent, and a portion of each of the spaces is defined by a defining member including movable regions each associated with a corresponding one of the spaces is employed. For example, the invention can be also applied to a color material ejection head for use in producing color filters for a liquid crystal display and the like; an electrode material ejection head for use in forming electrodes for an organic electro luminescence (EL) display, a field emission display (FED), or the like; and a living organic material ejection head for use in producing biotips (biochemical elements). For the color material ejection head for use in display producing apparatuses, a solution of each of color materials for red (R), green (G), and blue (B) colors is ejected as one kind of the liquid. Further, for the electrode material ejection head for use in electrode forming apparatuses, an electrode material in a liquid condition is ejected as one kind of the liquid, and for the living organic material ejection head for use in biotips producing apparatuses, a solution of a living organic material is ejected as one kind of the liquid.

What is claimed is:

1. A piezoelectric device comprising:
a substrate in which a plurality of spaces are arranged in parallel so as to be partitioned by a plurality of walls;
a defining member defining a portion of each of the spaces in such a way as to cross between adjacent walls being among the walls and corresponding to the each of the spaces on one face of the substrate; and
a plurality of piezoelectric elements formed in such a way as to be each associated with a corresponding one of the spaces on an opposite side of the defining member from a side of the spaces,
wherein, when a width of each of the walls in a direction in which the spaces are arranged in parallel is denoted by a sign a, a height of each of the walls is denoted by a sign b, the height being a size from the one face of the substrate up to another face of the substrate, the another face being on an opposite side of the substrate from the one face of the substrate, a thickness of the defining member is denoted by a sign t, and a long-length direction size of each of displaceable, movable regions in the defining member is denoted by a sign L, the width denoted by the sign a, the height denoted by the sign b, the thickness denoted by the sign t, and the size denoted by the sign L satisfy a formula, t×L⁴/(a×b³)≤5×10⁵.

2. The piezoelectric device according to claim 1, wherein the width denoted by the sign a, the height denoted by the sign b, the thickness denoted by the sign t, and the size denoted by the sign L further satisfy a formula, 1.2×10⁵≤t×L⁴/(a×b³)≤1.6×10⁵.

3. The piezoelectric device according to claim 1, wherein the width denoted by the sign a, the height denoted by the sign b, the thickness denoted by the sign t, and the size denoted by the sign L satisfy a formula, t×b⁴/(a×L³)≤1.2×10⁻³.

4. The piezoelectric device according to claim 1, wherein the defining member has a residual stress.

5. The piezoelectric device according to claim 4, wherein a base material of the walls is silicon, and the defining material is a silicon oxide film that is formed by thermally oxidizing the base material.

6. A liquid ejecting head comprising the piezoelectric device according to claim 1.

7. A liquid ejecting head comprising the piezoelectric device according to claim 2.

8. A liquid ejecting head comprising the piezoelectric device according to claim 3.

9. A liquid ejecting head comprising the piezoelectric device according to claim 4.

10. A liquid ejecting head comprising the piezoelectric device according to claim 5.

11. A liquid ejecting apparatus comprising the liquid ejecting head according to claim 6.

12. A liquid ejecting apparatus comprising the liquid ejecting head according to claim 7.

13. A liquid ejecting apparatus comprising the liquid ejecting head according to claim 8.

14. A liquid ejecting apparatus comprising the liquid ejecting head according to claim 9.

15. A liquid ejecting apparatus comprising the liquid ejecting head according to claim 10.
